(19) United States
(12) Patent Application Publication
(10) Pub. No.: US 2026/0201411 A1
(43) Pub. Date: Jul. 16, 2026

(54) Trivalent Tuberculosis Vaccine

(71) Applicant: McMaster University, Hamilton (CA)

(72) Inventors: Zhou Xing, Ancaster (CA); Sam Afkhami, Hamilton (CA)

(21) Appl. No.: 19/127,886

(22) PCT Filed: Nov. 14, 2023
(86) PCT No.: PCT/CA2023/051523

§ 371 Date: May 7, 2025

Related U.S. Application Data
(60) Provisional application No. 63/383,566, filed on Nov. 14, 2022.

(51) Int. Cl. C12N 15/86; A61K 39/00; A61K 39/04; A61P 37/04; C07K 14/35

(52) U.S. Cl. CPC C12N 15/86; A61K 39/04; A61P 37/04; C07K 14/35; A61K 2039/5256; A61K 2039/53; A61K 2039/544; A61K 2039/70; C12N 2710/10043

(57) ABSTRACT

A multi-valent vaccine is provided which targets the multi-stage life cycle of Mycobacterium species of the tuberculosis complex. The vaccine comprises a transgene that expresses an acute mycobacterial infection-associated antigen such as Ag85A, an acute/chronic mycobacterial infection-associated antigen such as TB 10.4, and a dormant/latent resuscitation mycobacterial infection-associated antigen such as RpfB, for induction of broad protective immunity against pulmonary tuberculosis. The vaccine provides robust mucosal immunity against not only actively replicating but also dormant non-replicating mycobacteria in the lung.

### FIELD OF THE INVENTION

The present invention generally relates to the field of vaccines, and in particular, to a novel vaccine for tuberculosis.

### BACKGROUND

Pulmonary tuberculosis (TB) remains a leading cause of global morbidity and mortality by a single infectious agent, accounting for 10 million cases and 1.5 million deaths annually with an estimated ¼ world population being latently infected (WHO). The END TB initiative led by the World Health Organization remains an integral strategy in combatting TB, in-part relying on enhanced disease surveillance, treatment access, and connected health networks in endemic regions. Although this initiative has globally had a positive impact in TB control, the emergence and continued spread of SARS-CoV-2 has reversed years of progress, as 2020 represented the first year with increased indices of TB disease and death. Despite TB being a global phenomenon, its highest burden is seen in TB-endemic regions that were heavily impacted by the COVID-19 pandemic. These findings highlight the importance of developing and refining strategies that can combat TB without the continuous dependence on lifelines that can be rapidly derailed.

Vaccination aims to generate long-lasting host immunity and is a critical pillar in global TB control and its ultimate elimination. Unfortunately, the only approved TB vaccine, Bacillus Calmette-Guerin (BCG), which has globally been administered via the skin shortly after birth for more than 7 decades, has failed to provide effective protection against adult pulmonary TB. Over the last couple of decades, there has been an intense global effort in developing novel vaccine strategies to boost BCG-primed immunity. In this regard, currently there are at least a dozen lead TB vaccine candidates at various stages of clinical evaluation. Among these current-generation vaccines are two candidates that have recently been evaluated for their protective efficacy in Phase 2b trials. Unfortunately, these trials either showed no enhancement in protection (MVA85A) or demonstrated only limited efficacy in a narrow cohort of participants (M72/ASO1_{E}).

Since some of the lead vaccine candidates currently in the pipeline are similar to these two candidates in vaccine design, it is doubtful that they will ultimately be successful. Of note, the vast majority of current-generation recombinant subunit and viral-vectored TB vaccines are limited in vaccine antigen coverage and are administered parenterally via the skin or into the muscle. One powerful way to improve vaccine efficacy is to deliver vaccine via the respiratory route for induction of robust protective respiratory mucosal immunity. In this regard, viral-vectored TB vaccines for delivery via the respiratory route have been developed and have provided supporting clinical evidence for their safety and immunogenicity following aerosol delivery to human lungs. However, these viral-vectored vaccines were designed to express only a single-stage M tb antigen, Ag85A, produced mostly during the acute stage of infection similar to several other subunit or viral-vectored TB vaccine candidates.

It has increasingly been recognized that the next-generation vaccine strategy needs to consider the life cycle of M.tb bacilli during infection (Andersen and Scriba, 2019; Gengenbacher and Kaufmann, 2012; Yousefi Avarvand et al., 2019). Upon infection, confronting the host's immune pressure and environmental stresses such as nutrient deprivation and oxygen depletion, M.tb bacilli metabolically shift from an actively replicating state to a quiescent state of persistence, becoming non-replicating persisters or dormant M.tb bacilli, a feature of latent TB. This process is associated with differential M.tb antigen (Ag) expression with some Ags such as Ag85 complex proteins (AgA/B/C) predominantly produced during the acute stage of infection, some including ESX secretion system proteins (TB10.4, ESAT6, etc.) produced during both the acute and chronic stages of infection, and some only expressed during the latent or dormant stage of infection, also named as the latency-associated Ags. Infection stage-dependent Ag expression represents one of the immune-evasive strategies for M.tb and it can render the current vaccines expressing only the acute stage Ags ineffective, particularly in host defense against chronic and latent TB (Moguche et al., 2017). Since the front-line TB antibiotics can only target the actively replicating M.tb bacilli, the latent TB and its reactivation represent the greatest challenge to TB control and vaccine development. Thus, a next-generation vaccine to address this issue is desirable (Aagaard et al., 2011a; Hansen et al., 2018; Ma et al., 2017; Xin et al., 2013).

Among the latency-associated M.tb Ags are the five resuscitation-promoting factors (rpfA/B/C/D/E) involved in resuscitation of dormant M.tb bacilli and TB reactivation. Humans with latent TB were found to harbor T cells strongly reactive to rpf antigens. Preclinical studies have further revealed that rpfB Ag is the most immunogenic of the five rpf Ags and represents a robust CD8 T cell activator. Although some of the latency-associated Ags including select rpf members have been included in multi-stage vaccine design (Aagaard et al., 2011a; Hansen et al., 2018; Ma et al., 2017; Xin et al., 2013), whether such vaccines have any impact on non-replicating dormant M tb bacilli still remains unknown. This is due to the fact that the conventional method to determine TB vaccine protective efficacy is via enumerating colony forming unit (CFU) of replicating bacilli cultured on solid agar which does not capture the non-replicating dormant M.tb. Furthermore, multi-stage TB vaccine design has not been incorporated into the respiratory mucosal immunization strategy with recombinant viral vectors.

Thus, there is a need for a novel multivalent vaccine for use against pathogens with a multi-stage life cycle in which antigen expression varies across the life cycle of the pathogen.

### SUMMARY

A multivalent tuberculosis vaccine has now been developed. This vaccine is designed to target the multi-stage life cycle of mycobacterial bacilli, such as Mycobacterium tuberculosis (M.tb), by expressing an acute mycobacterial infection-associated antigen, an acute/chronic mycobacterial infection-associated antigen, and a dormant/latent resuscitation mycobacterial infection-associated antigen for induction of broad protective immunity against pulmonary tuberculosis.

Thus, in one aspect, a transgene is provided that encodes a multivalent tuberculosis vaccine encoding an acute mycobacterial infection-associated antigen, an acute/chronic mycobacterial infection-associated antigen, and a dormant/latent resuscitation mycobacterial infection-associated antigen.

In another aspect, a trivalent vaccine is provided comprising a transgene that encodes an acute mycobacterial infection-associated antigen, an acute/chronic mycobacterial infection-associated antigen, and a dormant/latent resuscitation mycobacterial infection-associated antigen.

In another aspect of the invention, a method of vaccinating a mammal against a mycobacterial infection is provided comprising administering a trivalent vaccine comprising a transgene that encodes an acute mycobacterial infection-associated antigen, an acute/chronic mycobacterial infection-associated antigen, and a dormant/latent resuscitation mycobacterial infection-associated antigen, to a mammal.

In a further aspect, a trivalent vaccine comprising a transgene that encodes an acute mycobacterial infection-associated antigen, an acute/chronic mycobacterial infection-associated antigen and a dormant/latent resuscitation mycobacterial infection-associated antigen is provided for use to vaccinate a mammal against mycobacterial infection.

In embodiments of the invention, the trivalent vaccine is prepared for administration via the respiratory tract, and is administered by respiratory mucosal administration.

These and other aspects of the invention are described in the detailed description that follows by reference to the following Figures.

### BRIEF DESCRIPTION OF THE FIGURES

FIG. 1 illustrates transgene design and immunogenicity of a multi-stage ChAd:TB vaccine according to an embodiment of the invention, including: A) a schematic of a transgene cassette, Tri:ChAd:TB; B) transgene expression analysis by PCR using DNA isolated from A549 cells transduced with Tri:ChAd:TB; C) stacked bar graphs depicting the absolute number of CD8⁺ T cell responses in the BAL two weeks post-intranasal (i.n.) vaccination with either Mono:ChAd:TB or Tri:ChAd:TB, as measured by expression of IFNγ following ex vivo stimulation with Ag85A, TB10.4, or rpfB whole protein; D) representative flow cytometric plots of IFNγ⁺ CD8⁺ T cells in the BAL two weeks post-i.n. immunization with either Mono:ChAd:TB or Tri:ChAd:TB, following ex vivo stimulation with Ag85A, TB10.4, or rpfB whole protein; E) stacked bar graphs depicting the absolute number of CD8⁺ T cell responses in the lung two weeks post-i.n. immunization with either Mono:ChAd:TB or Tri:ChAd:TB, as measured by expression of IFN-γ following ex vivo stimulation with Ag85, TB10.4 or rpfB whole protein; F) representative flow cytometric plots of IFNγ⁺ CD8⁺ T cells in the lung two weeks post-i.n. immunization with either Mono:ChAd:TB or Tri:ChAd:TB, following ex vivo stimulation with Ag85A, TB10.4, or rpfB whole protein; G) stacked bar graphs depicting the absolute number of CD8⁺ T cell responses in the lung parenchymal tissue (LPT) six weeks post-i.n. immunization with either Mono:ChAd:TB or Tri:ChAd:TB, as measured by expression of IFN-γ following ex vivo stimulation with Ag85, TB10.4 or rpfB whole protein; H) pie charts depicting the functionality (IFNγ, TNF-α, and/or IL-2) of CD8+T cells six weeks post-i.n. immunization with either Mono:ChAd:TB or Tri:ChAd:TB, following ex vivo stimulation with Ag85A, TB10.4, or rpfB whole protein; and I) Left panel: t-SNE map generated from concatenating CD3⁺ CD8⁺ CD4⁻ gated cells from lung mononuclear cells from animals i.n-vaccinated with tri:ChAd:TB and stimulated with either Ag85A,TB10.4, or rpfB whole protein. Middle panel: Overlayed populations representing either Ag85A, TB10.4, or rpfB-specific antigen-specific T cells. Right panel: Heatmap projection of CD69⁺CD103⁺CD49a⁺ populations.

FIG. 2 illustrates immunogenicity of a multi-stage ChAd:TB vaccine in BCG-primed animals including: A) experimental schema; B) bar graphs depicting the absolute number of either CD4⁺ or CD8⁺ T cell responses in the BAL, as measured by expression of IFNγ following ex vivo stimulation with crude BCG/culture filtrate; C) pie charts depicting the functionality (IFNγ, TNF-α, and/or IL-2) of CD8⁺ or CD4⁺ T cells following ex vivo stimulation with crude BCG/culture filtrate; D) flow cytometric plots of IFNγ⁺ CD4⁺ T cells in the BAL from concatenating CD3⁺ gated cells following ex vivo stimulation with Ag85A, TB10.4, or rpfB whole protein; E) flow cytometric plots of IFNγ⁺ CD8⁺ T cells in the BAL from concatenating CD3⁺ gated cells following ex vivo stimulation with Ag85A, TB10.4, or rpfB whole protein; and F) bar graphs depicting the absolute number of either CD4⁺ (red) or CD8⁺ (blue) T cell responses in the spleen, as measured by expression of IFNγ following ex vivo stimulation with crude BCG/culture filtrate (data presented in (B and F) represent mean±SEM of n=3 mice/group. Statistical analysis for (B and F) were 2-way ANOVA with Tukey's multiple comparison test. *p<0.05; ****p<0.0001.

FIG. 3 illustrates the protective efficacy of a multi-stage ChAd:TB vaccine against M.tb (H₃₇Rv) in the BALB/c model, including: A) experimental schema, pertaining to B) and C; B) lung mycobacterial burden (log₁₀ colony forming unit (CFU)) four weeks post-Mtb H₃₇Rv challenge; C) representative lung histopathological images four weeks post-M.tb challenge. Black arrows indicate granulomatous lesions; D) transgene cassette diagram for Biv:ChAd:TB; E) experimental schema, pertaining to F); and F) lung mycobacterial burden (Log₁₀ CFU) four weeks post-Mtb challenge (data presented in (B and F) represent mean±SEM of n=3-5mice/group. Statistical analysis for (B and F) were 1-way ANOVA with Tukey's multiple comparison test. **p<0.01; ****p<0.0001.

FIG. 4 illustrates the protective efficacy of a multi-stage ChAd:TB vaccine against M.tb persisters in the BALB/c model, including: A) experimental schema, pertaining to panel B; B) lung mycobacterial burden (Log₁₀ colony forming unit (CFU)); C) diagram depicting generation of M.tb culture filtrates for assessment of mycobacterial persisters in lung homogenates; D) Left: Most Probable Number (MPN) estimates (log₁₀ ) to assess actively replicating bacilli (left bar of each pair—conventional media) and persisters (right bar of each pair—resuscitation media). Right: Resuscitation Index, as calculated by a ratio of persisters-to-actively replicating bacilli; E) experimental schema, pertaining to F); and F) Left: Most Probable Number (MPN) estimates (Log₁₀ ) to assess actively replicating bacilli (left bar of each pair—conventional media) and persisters (right bar of each pair—resuscitation media). Right: Resuscitation Index, as calculated by a ratio of persisters-to-actively replicating bacilli (data presented in (B, D and F) represent mean±SEM of n=3 mice/group. Statistical analysis for (B) was 1way ANOVA with Tukey's multiple comparison test. Statistical analysis for (D and F) was 2-way ANOVA with Tukey's multiple comparison test).

FIG. 5 illustrates protective efficacy of a multi-stage ChAd:TB vaccine against M.tb in the C3HeB/FeJ model, including: A) experimental schema, pertaining to B); B) survival curve following aerosol challenge with M.tb erdman; C) experimental schema, pertaining to panels D)-G); D) lung mycobacterial burden (log₁₀ colony forming unit (CFU)). A indicates change in CFU relative to the average from the control group; E) MPN estimates (log 1) to assess actively replicating bacilli (using conventional (conv.) media and persisters using resuscitation (resus.) media; F) Top: representative gross lung images 15 weeks post-M.tb challenge.. Bottom: Representative lung histopathological images 15 weeks post-M.tb challenge; and G) bar graphs depicting total number of gross lesions on the left lung (data presented in (D, F and G) represent mean±SEM of n=4-5 mice/group. Statistical analysis for (F) was 1way ANOVA with Tukey's multiple comparison test. ****p<0.0001).

FIG. 6 illustrates protective efficacy of a multi-stage ChAd:TB vaccine against M tb (H₃₇Rv) in the Hu-mouse model, including A) experimental schema; B) weight change curve following intranasal (i.n.) challenge with M.tb H₃₇Rv; C) lung mycobacterial burden (log₁₀ colony forming unit (CFU)); D) representative lung Acid Fast Bacilli (AFB) images; E) Top: Representative gross lung images. Bottom: Representative lung histopathological images; and F) scatter plots depicting percentage area of granulomatous tissue in lungs (data presented in (B, C, and E) represent mean±SEM of n=3-4mice/group. Statistical analysis for (C and E) were two-tailed T tests. **p<0.01).

FIG. 7 illustrates immunogenicity of a multi-stage ChAd:TB vaccine, including A) stacked bar graphs depicting the absolute number of CD4⁺ T cell responses in the BAL two weeks post-intranasal (i.n.) vaccination with either Mono:ChAd:TB or Tri:ChAd:TB, as measured by expression of IFNγ following ex vivo stimulation with Ag85A, TB10.4, or rpfB whole protein; B) representative flow cytometric plots of IFNγ⁺ CD4⁺ T cells in the BAL two weeks post-i.n. immunization with either Mono:ChAd:TB or Tri:ChAd:TB, following ex vivo stimulation with Ag85A, TB10.4, or rpfB whole protein; C) stacked bar graphs depicting the absolute number of CD4⁺ T cell responses in the lung two weeks post-i.n. immunization with either Mono:ChAd:TB or Tri:ChAd:TB, as measured by expression of IFN-γ following ex vivo stimulation with Ag85A, TB10.4, or rpfB whole protein; D) representative flow cytometric plots of IFNγ⁺ CD4⁺ T cells in the lung two weeks post-i.n. immunization with either Mono:ChAd:TB or Tri:ChAd:TB, following ex vivo stimulation with Ag85A, TB10.4, or rpfB whole protein; and E) Left: Experimental schema. Right: Representative flow cytometric plots depicting localization of immune cells in either the lung parenchymal tissue (LPT) or lung vasculature (data presented in (A-E) represent mean±SEM of n=3 mice/group).

FIG. 8 illustrates immunogenicity of a multi-stage ChAd:TB vaccine in BCG-primed animals, including A) bar graphs depicting the absolute number of either CD4⁺ (red) or CD8⁺ (blue) T cell responses in the lung, as measured by expression of IFNγ following ex vivo stimulation with crude BCG/culture filtrate; B) pie charts depicting the functionality (IFNγ, TNF-α, and/or IL-2) of CD8⁺ or CD4⁺ T cells following ex vivo stimulation with crude BCG/culture filtrate; C) flow cytometric plots of IFNγ⁺ CD4⁺ T cells in the BAL following i.n. immunization with either Mono:ChAd:TB or Tri:ChAd:TB, following ex vivo stimulation with Ag85A, TB10.4, or rpfB whole protein; and D) flow cytometric plots of IFNγ⁺ CD8⁺ T cells in the BAL following i.n. immunization with either Mono:ChAd:TB or Tri:ChAd:TB, following ex vivo stimulation with Ag85A, TB10.4, or rpfB whole protein (data presented in (A) represent mean±SEM of n=3 mice/group).

FIG. 9 illustrates amino acid sequences of acute mycobacterial infection-associated antigens A) Ag85A and B) Ag85B, and acute and chronic mycobacterial infection antigens C) TB10.4 and D) ESAT6.

FIG. 10 illustrates amino acid sequences of dormant/latent resuscitation antigens, A) RpfA, B) RpfB, C) RpfC, D) RpfD and E) RpfE.

### DETAILED DESCRIPTION OF EMBODIMENTS OF THE INVENTION

A trivalent vaccine is provided comprising a transgene encoding an acute mycobacterial infection-associated antigen, an acute/chronic mycobacterial infection-associated antigen, and a dormant/latent resuscitation mycobacterial infection-associated antigen.

The term “mycobacterial” as used herein with respect to antigens for use in the trivalent vaccine encompasses antigens which are obtained from a Mycobacterium species of the tuberculosis complex and includes those species traditionally considered as causing the disease tuberculosis, as well as Mycobacterium environmental and opportunistic species that cause tuberculosis and pulmonary disease in immune-compromised individuals (e.g. HIV-infected patients). Exemplary species of the tuberculosis complex for use herein include without limitation M tuberculosis (M.tb), M. bovis, M. bovis BCG, M africanum, M. canetti, M. caprae, and M. microti.

The trivalent vaccine is based on a transgene that encodes an antigen from each of the stages of the life cycle of a Mycobacterium species of the tuberculosis complex, including an antigen from the acute infection stage, an antigen from the acute/chronic stage, and an antigen from the dormant/latent resuscitation stage. This permits the trivalent vaccine to exhibit efficacy against Mycobacterium infection at any stage of its life cycle. Specifically, the vaccine will induce a strong, multifunctional immune response on administration against not only active, replicating mycobacteria but also non-replicating dormant mycobacteria.

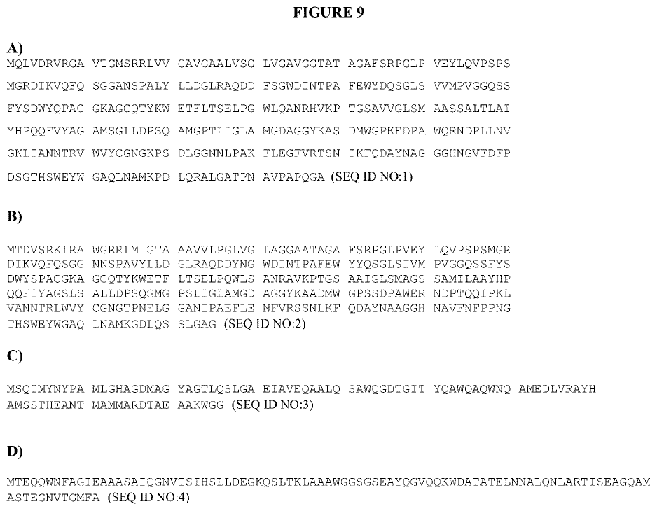

Antigens from the acute infection stage include, but are not limited to, the Ag85 complex proteins, such as Ag85A, Ag85B and Ag85C, as well as antigenic fragments thereof, e.g. fragments of the antigen which include one or more epitopes and thus, which are sufficient to induce an immune response. Antigenic fragments, thus, may be a truncated version of a full-length antigen from either end of the antigen, or an interior fragment of the antigen, and may additionally include amino acid modifications such as amino acid insertions, deletions or substitutions (such as, but not limited to, substitutions with conservative amino acids), wherein the insertion, deletion or substitution does not significantly adversely affect the immunogenicity of the fragment. Generally, antigenic fragments include modifications in a region or regions of an established antigen that does not include an epitope, and thus, such modifications are not expected to adversely affect immunogenicity/antigenicity. For example, antigenic fragments of Ag85A and Ag85B may include epitope-containing peptides spanning amino acids 100 to 120, amino acids 151 to 170, and 191 to 210 of Ag85A or Ag85B. The amino acid sequences of Ag85A and Ag85B are provided in FIG. 9 (A/B). A preferred acute infection antigen is Ag85A or an antigenic fragment thereof. Nucleic acid sequences for inclusion in the transgene are known based on the amino acid sequences of the antigen which are readily available on sequence databases, as are relevant gene and transcript sequences, for example, the comprehensive genomic and proteomic data repository for mycobacteria known as Mycobrowser.

The acute/chronic infection antigen comprises ESX secretion system proteins, including but not limited to, TB10.4, ESAT6, CFP-10, and Esp proteins such as EspC, as well as antigenic fragments thereof. The amino acid sequences of TB10.4 and ESAT6 are provided in FIG. 9 (C/D). A preferred acute/chronic infection antigen is TB10.4, or an antigenic fragment thereof. As above, nucleic acid sequence for inclusion in the transgene may be derived from the amino acid sequence of the antigen, or obtained from sequence databases.

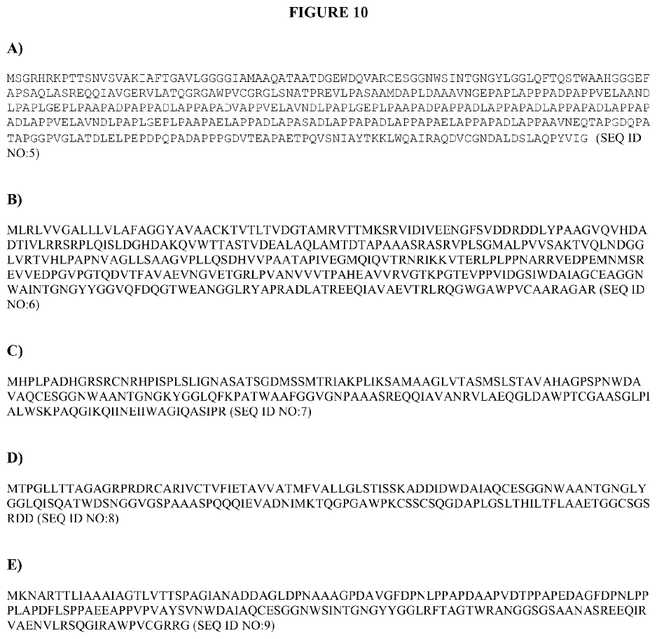

The dormant/latent resuscitation antigen comprises latency-associated Mycobacterial antigens such as the resuscitation-promoting factors (Rpf), RpfA, RpfB, RpfC, RpfD and RpfE, and antigenic fragments thereof. The amino acid sequences of the Rpf are provided in FIG. 10. A preferred dormant/latent resuscitation antigen is RpfB, or an antigenic fragment thereof. As above, nucleic acid sequence for inclusion in the transgene may be derived from the amino acid sequence of the antigen, or obtained from sequence databases.

A transgene encoding the selected antigens from each phase of the mycobacterial life cycle is prepared using techniques established in the art. The transgene incorporates nucleic acid encoding an N-terminal signal sequence comprising about 12-40 amino acids which, once translated, functions to translocate the antigens for secretion. The signal peptide is not particularly limited. Numerous signal peptides are used for production of secreted proteins, such as the protein expressed from the present transgene, including but not limited to, an immunoglobulin signal peptide such as murine immunoglobulin signal peptide (IgSP, EMBL Accession No. M13331), and signal peptides (leader sequences) from tissue plasminogen activator (tPA), insulin, Vesicular Stomatitis Virus glycoprotein (VSVG), IL-2, albumin, chymotrypsin and other immunoglobulins. Hybrid leader sequences have also been developed, for example, a leader sequence comprising an immunoglobulin signal peptide fused to a tissue-type plasminogen activator propeptide. Nucleic acid encoding the signal sequence is connected or linked to the antigen-encoding nucleic acid, and the nucleic acid encoding each antigen is linked with a short linker, e.g. nucleic acid encoding an amino acid repeat such a four glycines.

The transgene may be constructed for use in a nucleic acid-based vaccine, e.g. mRNA or DNA vaccine, or a viral-vectored vaccine.

For use in a DNA vaccine, the transgene coding regions for the antigen components, i.e. an antigen from the acute infection stage, an antigen from the acute/chronic stage and an antigen from the dormant/latent resuscitation stage, may be synthesized, or otherwise obtained, for example commercially, and linked, as described, with DNA encoding an amino acid repeat sequence. The vaccine construct will incorporate a nucleic acid encoding a signal sequence as described, a suitable promoter to regulate expression of the transgene, as well as a transcriptional stop sequence, e.g. a poly (A) addition sequence, at the terminal end thereof. Examples of suitable promoters for incorporation in the vaccine construct, include but are not limited to, CMV, EF1a, CAG, PGK1, SV40, RSV, TRE, U6, UAS, Ubc, human beta actin, and CAG. The construct may also incorporate enhancer elements and/or transcriptional transactivators to enhance promoter activity when placed either upstream or downstream of the ORF.

The DNA transgene construct is then generally adapted for administration. The transgene construct may be formulated for administration as a linear molecule, covalently-closed linear construct or mini-circle. Alternatively, the transgene construct may be incorporated into a vector such as a plasmid or cosmid using techniques well-known in the art. The resulting DNA vaccine is then formulated for administration. The vaccine may be incorporated into a delivery system adapted to enhance immunogenicity of the vaccine, for example, biodegradable polymeric microparticles (e.g. chitosan, polylactic-co-glycolides, polyethyleneimine, amine-functionalized polymethacrylates, cationic poly(O-amino esters), poloxamers and polyvinylpyrrolidone polymers) or liposomes. The vaccine may also be combined with an adjuvant to enhance immunogenicity, e.g. inorganic compounds such as aluminum-containing compounds, squalene, oils such as paraffin, bacterial products such as toxoids, plant saponins, cytokines such as IL-1, IL-2 or IL-12, a cytosine phosphoguanine (CpG) motif-containing adjuvant, or an adjuvant combination such as Freund's adjuvant.

For use in an mRNA vaccine, the DNA transgene construct is prepared as described, and mRNA is synthesized therefrom by in vitro transcription of the cDNA template, typically, plasmid DNA (pDNA), prepared as described using methods known in the art. Transcription of the cDNA template is conducted using RNA polymerase such as a bacteriophage RNA polymerase. For stability and efficient translation, the resultant mRNA strand will include a 5′ cap and 3′ poly(A) tail, as well as 5′ and 3′ untranslated regions (UTRs) flanking the coding region. The mRNA vaccine is then formulated for administration. In this regard, mRNA may be complexed with adjuvant agents which prevent degradation, enhance uptake and promote translation. Examples of such adjuvants include, but are not limited to, cationic polypeptides (e.g. protamine); polymer delivery systems such as spermine, polyethyleneimine, chitosan, polyurethane, poly-amido-amine (PAA), poly-beta amino-esters (PBAEs) and polyethylenimine (PEI); nanoemulsions, carrier peptides, liposomes, lipid nanoparticles, and immune activator proteins (e.g. CD70, CD40L, TLRs).

Viral-vectored vaccines may also be utilized to administer the present transgene construct, including both DNA viral vectors and RNA viral vectors. Such viral vector are suitable for administration to a mammal, i.e. do not themselves result in an unacceptable adverse effect, and are effective to deliver and express the transgene on administration to a mammal.

DNA viral vector vaccines are adapted to expressibly incorporate the present DNA transgene construct, e.g. under the control of a viral promoter. Examples of suitable DNA viruses for use as vaccines include, but are not limited to, poxviruses such as vaccinia virus and modified vaccinia virus, adenoviruses, adeno-associated viruses, herpes simplex virus and cytomegalovirus, and including various serotypes thereof, both replication-competent and replication-deficient or replication-incompetent.

In one embodiment, a replication-incompetent adenovirus is prepared for use to deliver the present transgene construct, for example, a human or chimpanzee adenoviral vector. In one embodiment, the transgene is incorporated within an E1/E3 region of the virus which has been deleted from the adenovirus.

RNA viral vector vaccines may also be adapted to expressibly incorporate an appropriate transcript of the present transgene construct, e.g. positive or negative strand. Examples of suitable RNA viruses for use as a vaccine to deliver the transgene include, but are not limited to, vesicular stomatitis viruses, retroviruses such as MoMLV, lentiviruses, Sendai viruses, measles-derived vaccines, Newcastle disease virus, alphaviruses such as Semliki Forest virus, flaviviruses, or an RNA replicon based on an RNA virus (i.e. derived from alphavirus, flavivirus, etc).

A protein vaccine may also be employed comprising an acute mycobacterial infection-associated antigen, a chronic mycobacterial infection-associated antigen, and a dormant/latent resuscitation mycobacterial infection-associated antigen, each as previously described. As one of skill in the art will appreciate, protein vaccines are generally administered in conjunction with an adjuvant, a substance that acts to accelerate, prolong, augment or enhance the immune response. Examples of adjuvants that may be used to augment the effects of a protein vaccine include, but are not limited to, inorganic compounds such as potassium alum, aluminium hydroxide, aluminium phosphate and calcium phosphate hydroxide; and organic substances including oils such as paraffin oil; bacterial products such as: toxoids or killed bacteria, for example, B. pertussis or M bovis: plant saponins; cytokines such as IL-1, IL-2 and IL-12; and squalene.

The present vaccine comprising a trivalent transgene, or a protein product thereof, as described herein, is used in a method of vaccinating a mammal against a mycobacterial infection, such as infection by M tuberculosis (M.tb). The vaccine is administered to the mammal in a prophylactically effective amount, i.e. an amount sufficient to generate in the mammal an immune response to prevent a mycobacterial infection. The vaccine may also be administered in a therapeutically effective amount, i.e. an amount sufficient to treat a mycobacterial infection which is administered subsequent to infection by a mycobacterium. The term “mammal” is used herein to encompass both human and non-human mammals, such as cats, dogs, mice, rats and other rodents, pigs, goats, sheep, cattle, horses, and the like. The mammal may or may not have been previously vaccinated with Bacillus Calmette-Guerin (BCG), i.e. the present vaccine may function as a booster to BCG vaccination. As one of skill in the art will appreciate, the amount required to generate an immune response will vary with a number of factors, including, for example, the particular transgene/antigens in the vaccine, the vector used to deliver the vaccine, and the mammal to be treated, e.g. species, age, size, etc. In this regard, for example, administration of a dosage in the range of about 10⁶ to 10⁸ pfu of adenoviral vector to a mouse is sufficient to generate an immune response. A corresponding amount will generally be sufficient for administration to a human or other mammal to generate an immune response.

With respect to use of the present trivalent vaccine as a booster to BCG vaccination, the trivalent vaccine is administered subsequent to the BCG vaccination, for example, ranging from about 1 week up to about 6 months. Preferably, the trivalent vaccine booster is administered within about 4-8 weeks, or 1-2 months, following BCG vaccination.

Use of the present vaccine in a therapeutic sense may be administered alone or in conjunction with an antibiotic. Examples of antibiotics useful to treat a mycobacterial infection or tuberculosis (TB) include, but are not limited to, isoniazid, rifampin (Rifadin, Rimactane), ethambutol (Myambutol) and pyrazinamide. Drug-resistant TB may be treated with a combination of antibiotics such as fluoroquinolones with amikacin or capreomycin (Capastat). To impede drug resistance, the selected therapy may be administered together with a drug such as bedaquiline (Sirturo) and linezolid (Zyvox).

The present vaccine is administered to a mammal to prevent or treat a mycobacterial infection using any one of several administrable routes including, but not limited to, parenteral administration such as intramuscular, intravenous, subcutaneous or intrathecal; or respiratory administration including intranasally or by inhalation. For nucleic acid-based vaccines, other techniques such as administration by electroporation or using gene gun technology may be utilized. The prime and boosting vaccines, which may be the same or different vaccine type (e.g. both the prime and boosting vaccine may be a nucleic acid-based vaccine, or both may be a viral vectored vaccine, or the prime vaccine may be nucleic acid-based and the boosting vaccine may be a viral vector, or vice versa), may be administered by the same or different administrable routes. As will be appreciated by one of skill in the art, the vaccine, and any adjuvants, are administered in a suitable carrier, such as saline or other suitable buffer.

In one embodiment, the present vaccine comprising a trivalent transgene as herein described is advantageously formulated for respiratory administration intranasally or by inhalation to directly target the respiratory mucosa. Preferably, the vaccine is provided as a viral-vectored vaccine in saline or other suitable buffer, to be nebulized to form a liquid aerosol for inhalation by mouth or via a nasal spray. In one embodiment, the vaccine is an adenoviral-vectored vaccine, e.g. huAd or ChAd. In another embodiment, the vaccine may be provided in kit form along with a nebulizer to assist with its administration.

Using this method, e.g. administration intranasally or by inhalation, the present trivalent vaccine is efficiently delivered to the respiratory mucosa to generate immunological memory within the lungs, the target organ of a respiratory infection by mycobacterium spp. such as Mycobacterium tuberculosis.

Thus, the trivalent vaccine is effective to induce a strong, multifunctional immune response on the respiratory mucosa, as well as remotely, against not only active, replicating mycobacteria but also non-replicating dormant mycobacteria (persisters). It is further noted that use of the present trivalent vaccine either alone or to boost a BCG primed host significantly enhances immune responses including multi-stage antigen-specific immune (CD4+ and CD8+T cell) responses and BCG-immune response, on the respiratory mucosal surfaces and at peripheral sites, a result that was not achievable using a monovalent vaccine (i.e. a vaccine including a single antigen). This translates into a significant reduction of bacterial load, e.g. at least about 1-2 log reduction, or at least a 2-fold reduction, and a greater reduction of bacterial load in BCG primed hosts (e.g. 2-3 log reduction, or at least 3-fold reduction of bacterial load or greater). The trivalent vaccine also significantly hinders growth of persistors, resulting in a significant decrease in resuscitation index (RI) (relative abundance of persisters-to-actively replicating mycobacteria) to an RI of 2 or less, such as 1 or less or less than 1, i.e. essentially abolishes persistors, as compared to greater RIs with BCG treatment alone or with vaccines that do not express a latency-associated antigen such as a resuscitation-promoting factor.

It is also noted that treatment of a mycobacterium-infected mammal with the present trivalent vaccine, either alone or as a BCG boost, results in increased survival time as compared to no treatment or treatment with BCG only, of at least about 10%, 15% or more.

Embodiments of the invention are described by reference to the following specific example which is not to be construed as limiting.

### Example 1

A trivalent viral-vectored vaccine against M. tuberculosis (M.tb) was prepared and tested using the following methods.

Molecular construction and validation of trivalent chimpanzee adenovirus vaccine—A replication-deficient chimpanzee serotype 68 adenovirus was constructed to express three M tb antigens—Antigens 85A, TB10.4, and rpfB using the following sequences:

```
Ag85A:
  (SEQ ID NO: 10)
atgcagcttgttgacagggttcgtggcgccgtcacgggtatgtcgcgtcgactcgtggtcggggccgtcggcgcggc
cctagtgtcgggtctggtcggcgccgtcggtggcacggcgaccgcgggggcattttcccggccgggcttgccggtgg
agtacctgcaggtgccgtcgccgtcgatgggccgtgacatcaaggtccaattccaaagtggtggtgccaactcgccc
gccctgtacctgctcgacggcctgcgcgcgcaggacgacttcagcggctgggacatcaacaccccggcgttcgagtg
gtacgaccagtcgggcctgtcggtggtcatgccggtgggtggccagtcaagcttctactccgactggtaccagcccg
cctgcggcaaggccggttgccagacttacaagtgggagaccttcctgaccagcgagctgccggggggctgcaggcc
aacaggcacgtcaagcccaccggaagcgccgtcgtcggtctttcgatggctgcttcttcggcgctgacgctggcgat
ctatcacccccagcagttcgtctacgcgggagcgatgtcgggcctgttggacccctcccaggcgatgggtcccaccc
tgatcggcctggcgatgggtgacgctggcggctacaaggcctccgacatgtggggcccgaaggaggacccggcgtgg
cagcgcaacgacccgctgttgaacgtcgggaagctgatcgccaacaacacccgcgtctgggtgtactgcggcaacgg
caagccgtcggatctgggtggcaacaacctgccggccaagttcctcgagggcttcgtgcggaccagcaacatcaagt
tccaagacgcctacaacgccggtggcggccacaacggcgtgttcgacttcccggacagcggtacgcacagctgggag
tactggggcgcgcagctcaacgctatgaagcccgacctgcaacgggcactgggtgccacgcccaacaccgggcccgc
gccccagggcgcctag
TB10.4:
(SEQ ID NO: 11)
atgtcgcaaatcatgtacaactaccccgcgatgttgggtcacgccggggatatggccggatatgccggcacgctgca
gagcttgggtgccgagatcgccgtggagcaggccgcgttgcagagtgcgtggcagggcgataccgggatcacgtatc
aggcgtggcaggcacagtggaaccaggccatggaagatttggtgcgggcctatcatgcgatgtccagcacccatgaa
gccaacaccatggcgatgatggcccgcgacacggccgaagccgccaaatggggcggctag
rpfB:
  (SEQ ID NO: 12)
atgttgcgcctggtagtcggtgcgctgctgctggtgttggcgttcgccggtggctatgcggtcgccgcatgcaaaac
ggtgacgttgaccgtcgacggaaccgcgatgcgggtgaccacgatgaaatcgcgggtgatcgacatcgtcgaagaga
acgggttctcagtcgacgaccgcgacgacctgtatcccgcggccggcgtgcaggtccatgacgccgacaccatcgtg
ctgcggcgtagccgtccgctgcagatctcgctggatggtcacgacgctaagcaggtgtggacgaccgcgtcgacggt
ggacgaggcgctggcccaactcgcgatgaccgacacggcgccggccgcggcttctcgcgccagccgcgtcccgctgt
ccgggatggcgctaccggtcgtcagcgccaagacggtgcagctcaacgacggcgggttggtgcgcacggtgcacttg
ccggcccccaatgtcgcggggctgctgagtgcggccggcgtgccgctgttgcaaagcgaccacgtggtgcccgccgc
gacggccccgatcgtcgaaggcatgcagatccaggtgacccgcaatcggatcaagaaggtcaccgagcggctgccgc
tgccgccgaacgcgcgtcgtgtcgaggacccggagatgaacatgagccgggaggtcgtcgaagacccgggggttccg
gggacccaggatgtgacgttcgcggtagctgaggtcaacggcgtcgagaccggccgtttgcccgtcgccaacgtcgt
ggtgaccccggcccacgaagccgtggtgcgggtgggcaccaagcccggtaccgaggtgcccccggtgatcgacggaa
gcatctgggacgcgatcgccggctgtgaggccggtggcaactgggcgatcaacaccggcaacgggtattacggtggt
gtgcagtttgaccagggcacctgggaggccaacggcgggctgcggtatgcaccccgcgctgacctcgccacccgcga
agagcagatcgccgttgccgaggtgacccgactgcgtcaaggttggggcgcctggccggtatgtgctgcacgagcgg
gtgcgcgctga
```

Each antigen is separated by linkers composed of 4 glycine residues (Tri:ChAd:TB), using previously described technology (Jeyanathan et al., 2015. Mucosal Immunol 8, 1373-1387). The transgene cassette was cloned to express the murine cytomegalovirus promoter (MCMV), and a tissue plasminogen activator peptide signal. Apart from the expressed antigens, this trivalent vector is molecularly identical to monovalent vector (Mono:ChAd:TB) expressing Ag85A alone. Briefly, a pShuttle plasmid was engineered to express the transgene cassette and amplified in DH5a E. coli (ThermoFisher, Waltham, MA, USA). The entire transgene cassette was excised and subcloned into the DNA clone pAdCh68 (ΔE1/E3) by I-Ceu1/PI-Sce1 digest and subsequent in-gel ligation. Trivalent AdCh68 was subsequently packaged and propagated in HEK 293 cells and purified by cesium chloride centrifugation. Transgene expression was validated by PCR on the supernatants and lysates of infected A549 mammalian cells.

Animal models for in vivo studies—Female BALB/c mice (6-8 weeks old) were purchased from Charles River (Wilmington, MA, USA). Female C3HeB/FeJ mice (6-8 weeks old) were purchased from The Jackson Laboratory (Bar Harbor, ME, USA). All experimental mice were housed within either the level 2 or level 3 containment facility at McMaster University, with all experiments being conducted in accordance with the McMaster University Animal Research Ethics Board.

Development and immune screening of Hu-mice—NOD-Rag1null IL2rgnull (NRG) mice were obtained from Jackson laboratories (Bar Harbor, Maine, USA). NRG pups (24-72 hours old) were irradiated twice with 3 cGy 3 hours apart and then engrafted intrahepatically with 10⁵-10⁶ CD34⁺ hematopoietic stem cells (HSC) isolated from human umbilical cord blood. At 12 weeks post-engraftment, blood was collected to quantify human immune cell reconstitution using flow cytometry. Erythrocytes were lysed using an ACK lysis buffer and the remaining cells were treated with both anti-human Fc Receptor Binding Inhibitor and anti-mouse CD16/CD32 antibodies (eBiosciences). Cells were then stained with an antibody cocktail (mCD45-AlexaFluor 700, hCD45-Pacific Blue, hCD3e-Qdot 605, hCD4-PerCP-Cy5.5, hCD8a-PE-Cy7), followed by fixable viability dye (APC-eFluor 780; eBiosciences). Samples were run on the Cytoflex LX flow cytometer equipped with a flow rate calibrator and analysed using FlowJo software version 10 (Tree Star, Ashland, OR, USA). Mice with at least 10% or 50,000 per mL hCD45+ leukocytes in the blood were selected for subsequent experiments.

Intranasal immunization with Tri:ChAd vaccines—Immunization was done by intranasal instillation of 1×10⁷ PFU of ChAd:TB vaccines in a total volume of 25 μL of sterile phosphate buffered saline (PBS). In select experiments, BCG (Pasteur) immunization was performed subcutaneously at a dose of 1×10⁵ CFU in 100 μL of sterile PBS.

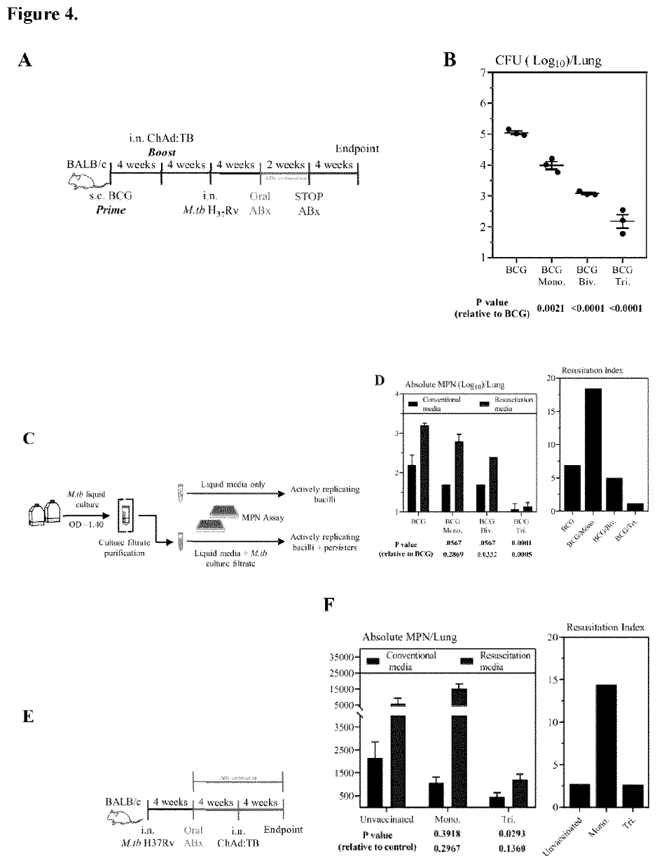

M. tuberculosis infection and antibiotic therapy—Mice were infected via the respiratory mucosal route with either 1×10⁴ colony-forming units of M. tuberculosis H37Rv (ATCC27294), or M. Tuberculosis erdman strain (kindly provided by Dr. David Russell), in a total volume of 25 μL of sterile PBS. In select experiments, infection was performed by aerosol inhalation utilizing a Glas-Col inhalation exposure system (Glas-Col, LLC, Terre Haute, IN, United States). Mice receiving antibiotics (ABx) for therapeutic immunization studies received them as an oral Medidrop (Clear H2O, Westbrook, ME, USA) solution of rifampicin (10 mg/kg), isoniazid (25 mg/kg) and/or pyrazinamide (150 mg/kg) (see FIG. 4A).

Mononuclear cell isolation—Lung and BAL mononuclear cells were isolated as previously described. Briefly, lungs were cut into small pieces and digested with 150 units of collagenase type 1 (Life Technologies, Grand Island, NY, USA) in RPMI medium at 37° C. with agitation for an hour. Digested lung pieces were then crushed through a 100 μm filter and red blood cells were removed by treatment with an ACK lysis buffer. BAL cells were isolated by centrifugation. Splenic mononuclear cells were isolated by crushing the organ through a 100 μm filter, with red blood cells being removed by treatment with an ACK lysis buffer. Cells were resuspended in RPMI supplemented with 10% FBS, 1% pen-strep, 1% L-glutamine.

Cell stimulation, intracellular cytokine staining, and flow cytometry—Mononuclear cells were cultured in U-bottom plates at a concentration of 20 million cells per mL. For stimulation, 5ug/well of either recombinant Ag85A, rpfB, and/or TB10.4 were used. Cells were stimulated for a total of 6 hours in the presence of brefeldin A (5 mg/mL; BD Pharmingen, San Jose, CA, USA). In select experiments, BCG-specific immune responses were assessed by stimulation with crude BCG and M.tb culture filtrate at a concentration of 1 ug/mL, in the presence of brefeldin A. Following incubation, cells were washed and blocked with CD16/CD32 FcBlock in 0.5% bovine serum albumin/PBS for 15 minutes on ice, prior to being stained with the experimental-specific flourochrome-labelled monoclonal antibodies, according to the manufacturer's instructions (BD Pharmingen). This included: T cell panel—CD3-V450, CD8a-PE-Cy7, CD4-APC-Cy7, IFN-γ-APC, TNFα-FITC, PE-IL2. Neutrophil panel—CD45-APC-Cy7, CD11b-PE-Cy7, and Ly6G-BV605 (all from BD Biosciences, San Jose, CA, USA). All flow cytometry data were collected using a Fortessa Cytometer and FACSDiva software (BD Biosciences, San Jose, CA, USA) and analyzed using FlowJo software version 10 (Tree Star, Ashland, OR, USA).

Measurement of tuberculosis disease outcome—Lung bacillary load was assessed by plating serially-diluted lung homogenates on Middlebrook 7H10 agar plates supplemented with 10% OADC growth supplement (BD Biosciences, San Jose, CA, USA), 5 μg/mL ampicillin and 50 μg/mL cycloheximide (Sigma-Aldrich, St. Louis, MO, USA). Lung pathology was assessed histologically using lungs embedded in paraffin and assessed following either hematoxylin and eosin (H&E) or Ziehl-Neelson acid-fast staining. Histological samples were visualized by the Zeiss M2 Imager System (Zeiss, Toronto, ON, Canada). Pulmonary inflammation was determined qualitatively by using Image J software (NIH, http://rsb.info.nih.2ov/nih-imae/) by measuring the areas of dense inflammatory infiltrates relative to the total lung sample area. For each animal, 3 independent lung slices were measured prior to being averaged.

Mycobacterial resuscitation assay—Lung homogenates from infected animals were cultured in flat bottom plates in either a conventional media (7H9 media supplemented with 10% (vol/vol OADC (BD Biosciences) and 0.05% Tween80) or a resuscitation media. Resuscitation media was extracted from culture supernatants isolated from M tb cultures grown in supplemented 7H9 media to mid-exponential stage (OD_{600 nm} 1.4). Briefly, bacteria were removed by centrifugation prior to being filtered twice through a 0.2 um filter. Resuscitation media was composed of 50% of conventional media (vol/vol). Media was supplemented with polymyxin B (200U/mL), carbenicillin (100ug/mL), trimethoprim (20ug/mL), and amphotericin B (10ug/mL). Most probable numbers were calculated as described (Jarvis et al., 2010. J Appl Microbiol 109, 1660-1667).

Statistical analysis—Two-tailed Student t tests for comparison between 2 groups. 1-way analysis of variance followed by post-test Tukey analysis for multiple-group comparison using GraphPad Prism 8 software (Version 8, La Jolla, CA, USA). Results were considered significant for P values≤0.05. Area-under-curve (AUC) analysis was done to summate changes in bacterial burden over time. Unpaired t tests were performed using AUC analysis.

### Results

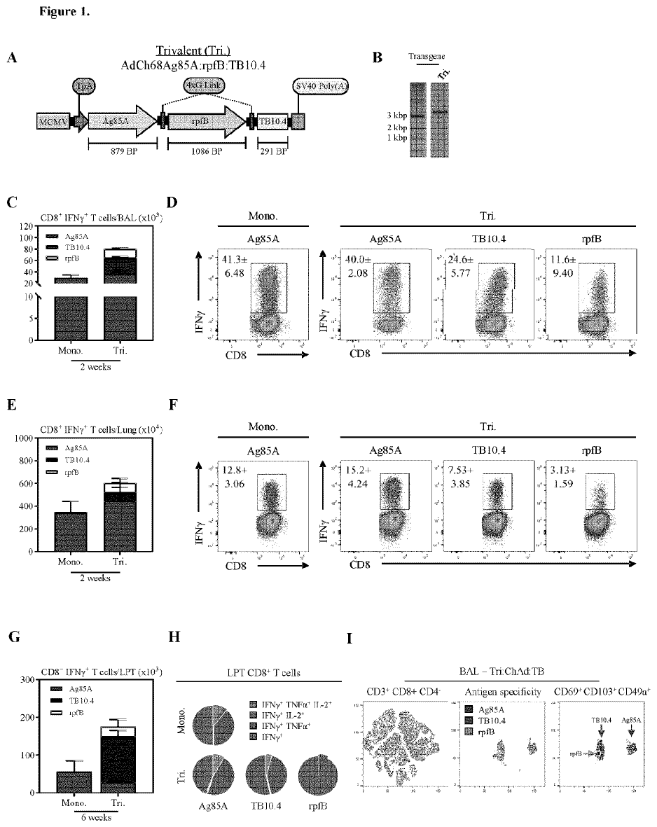

Molecular construction and characterization of a multi-stage ChAd:TB vaccine—The Tri:ChAd:TB vaccine was molecularly constructed utilizing the chimpanzee adenovirus serotype 68 backbone through a direct-cloning method. The transgene cassette was designed to express Ag85A, TB10.4, and rpfB, as a single transcript, with transgene expression under the control of the murine cytomegalovirus (MCMV) promoter, and transgene secretion by a human tissue plasminogen signal peptide sequence (TpA) (FIG. 1A). Ag85A and TB10.4 were cloned to flank rpfB with the use of flexible glycine (gly) linkers. Prior to viral rescue, the transgene cassette was sequence verified by Sanger sequencing. Tri:ChAd:TB was rescued through packaging and propagation in HeK 293 cells and purified by cesium chloride banding. Transgene expression was verified by polymerase chain reaction following transduction of A549 cells. In line with the expected size of the transgene cassette (FIG. 1A), a DNA band at approximately 3000BP was observed (FIG. 1B). A previously described monovalent chimpanzee adenovirus-vectored vaccine expressing Ag85A (Mono:ChAd:TB) developed in a similar fashion to Tri:ChAd:TB was utilized for downstream immunogenicity and efficacy experiments.

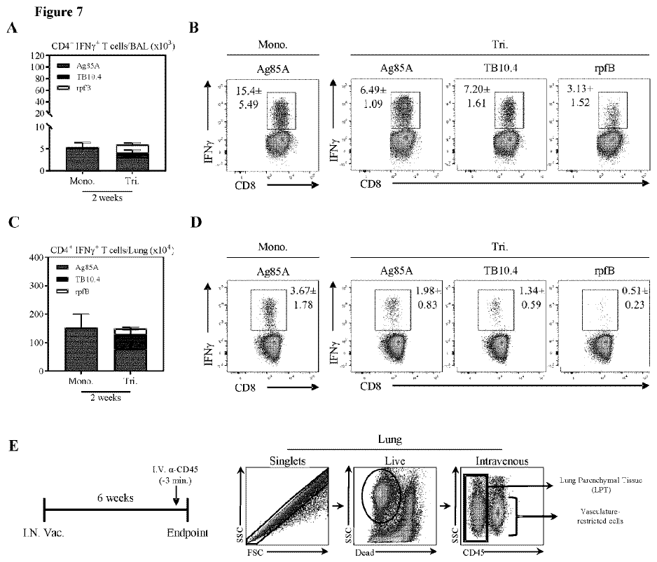

Intranasal immunization with multi-stage ChAd:TB vaccine induces antigen-specific T cell responses in the airways and lung tissues—The T cell immunogenicity of Tri:ChAd:TB vaccine was first characterized following respiratory mucosal immunization. Mice were vaccinated intranasally (i.n.) with a single dose of Tri:ChAd:TB vaccine (Tri). For comparison, a group of mice were i.n. immunized with an equal dose of its monovalent counterpart, Mono:ChAd:TB (Mono). T cell responses were analyzed in the airway lumen, represented by bronchoalveolar lavage (BAL) and lung tissue 2 weeks post-immunization. Vaccine encoded antigen-specificity of T cell responses were quantified by intracellular cytokine staining (IFNγ⁺) and flow cytometry following ex vivo stimulation with either recombinant Ag85A, TB10.4, or rpfB proteins. Intranasal Tri:ChAd:TB immunization induced high levels of Ag85A-, TB10.4- and rpfB-reactive CD8⁺ T cells in the airway lumen (FIG. 1C/D) and lung tissue (FIG. 1E/F). Notably, Ag85A-specific CD8⁺ T cells were greater in magnitude compared to TB10.4- and rpfB-specific CD8⁺ T cells both in the BAL and lung. Importantly, Ag85A-specific responses induced by Tri:ChAd:TB were comparable to the levels of Ag85A-specific responses induced by monovalent counterpart, indicating no antigenic competition despite inclusion of two additional antigens in Tri:ChAd:TB. Intranasal Tri:ChAd:TB immunization also induced Ag85A-, TB10.4- and rpfB-specific CD4⁺IFNγ⁺ T cells in the airway lumen (FIG. 7A/B) and lung tissue (FIG. 7C/D), but to a lesser degree than CD8⁺ T cell responses. These data suggest that the multi-stage ChAd:TB vaccine is capable of inducing robust T cell responses at the respiratory mucosal surfaces and in the lung against all three antigens encoded in the vaccine.

Intranasal immunization with multi-stage ChAd:TB vaccine induces multifunctional tissue-resident memory T cells—Since long lasting multifunctional-tissue-resident (T_{RM}) Ag-specific T cells at respiratory mucosal surfaces induced following vaccination are critical for effective host defense against pulmonary TB, the longevity, functionality and phenotypic characteristics of Ag-specific T cells induced following Tri:ChAd:TB vaccination were next evaluated and compared that to Ag-specific T cells induced by the monovalent counterpart vaccine. Mice were i.n. vaccinated with either Tri:ChAd:TB or ChAdAg85A and T cell responses in the airway lumen (BAL) and lung parenchymal tissue (LPT) were assessed 6 weeks post-immunization. Bona fide T cells within LPT were differentiated from intravascular counterparts (LV) via intravascular CD45.2 immunolabelling (Jeyanathan et al., 2017). To profile the multifunctionality of CD8⁺ T cell responses, total BAL and lung mononuclear cells were ex vivo stimulated with congruent recombinant proteins as described above and subjected to intracellular cytokines staining for IFNγ, TNFα, and IL-2. A sizeable population of Ag85A-, TB10.4- and rpfB-specific CD8+IFN γ+ T cells still remained in the LPT 6 weeks post-Tri:ChAd:TB immunization (FIG. 1G). Interestingly, proportions of Ag85A-, TB10.4- and rpfB-specific CD8⁺IFNγ⁺ T cells at 6 weeks post-immunization differed considerably from the proportions at 2 weeks post-immunization. While Ag85A-specific CD8⁺IFNγ⁺ T cells predominated during the effector phase (FIG. 1E), TB10.4-specific CD8⁺IFNγ⁺ T cells predominated during the memory phase (FIG. 1G). In keeping with effector phase, magnitude of Ag85A-specific CD8⁺IFNγ⁺ T cell responses were comparable between Tri:ChAd:TB and Mono:ChAd:TB-immunized hosts during the memory phase (FIG. 1G). Functionally, the majority of Ag85A- and TB10.4-specific CD8 T cells were bi-(IFNγ⁺ TNFα⁺) or mono-(IFNγ+) cytokine producers in Tri:ChAd:TB vaccinated hosts (FIG. 1H). In contrast rpfB-specific CD8+T cells were primarily monofunctional, solely producing IFNγ. Ag85A-specific CD8 T cells induced by Tri:ChAd:TB and Mono:ChAd:TB were functionally similar (FIG. 1H).

Having established that i.n. Tri:ChAd:TB vaccine induces long-lasting multifunctional antigen-specific CD8+ T cells within the LPT, the expression of resident memory surface markers CD69, CD103 and CD49a by Ag85A-, TB10.4- and rpfB-specific CD8⁺IFNγ⁺ T cells were profiled using t-SNE analysis on concatenated CD3⁺CD8⁺CD4⁻ BAL mononuclear cells from Tri:ChAd:TB vaccinated animals (FIG. 1I, left panel). Within the t-SNE map of total BAL CD8+T cells, overlayed congruent Ag-specific CD8+T cells clustered into three unique populations representing Ag85A-, TB10.4-, and rpfB-specific cells (FIG. 1I, middle panel). Expression of resident markers CD69, CD103 and CD49a by t-SNE analysis identified majority of Ag-specific cells being resident memory CD8⁺ T cells (FIG. 1I, right panel).

Collectively, the above data indicates that CD8⁺ T cells reactive to multi-stage antigens induced by Tri:ChAd:TB immunization is sustained on the mucosal surfaces with multiple functionality and acquire bono fide lung resident memory phenotype.

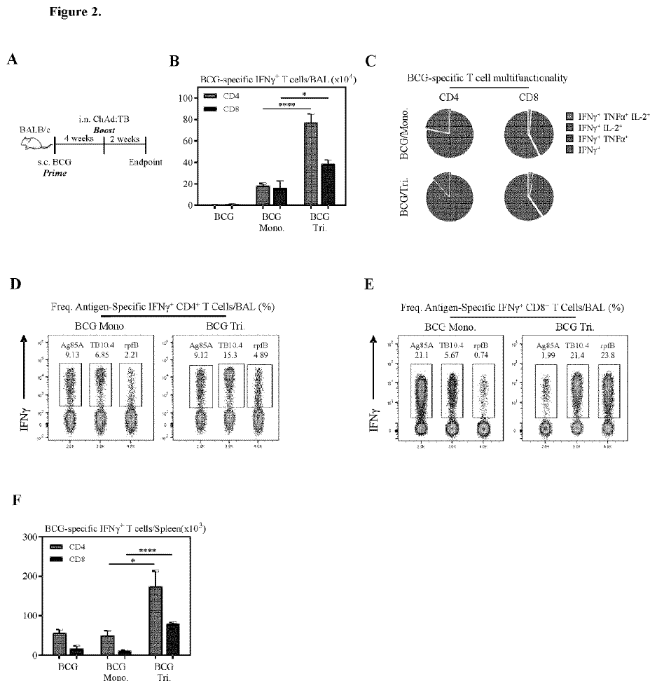

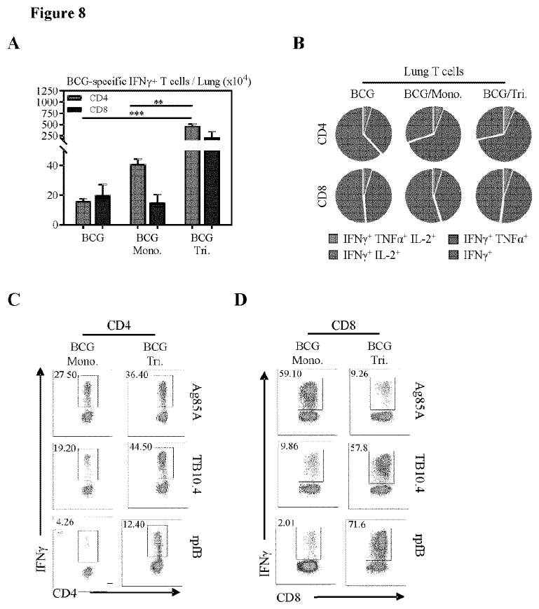

Intranasal vaccination with multi-stage ChAd:TB markedly boosts antigen-specific T cell responses in parenteral BCG-primed hosts—Despite being less efficacious against adult pulmonary forms of TB, the effectiveness of BCG against disseminated childhood disease makes it a foundation of the global immunization program for TB. As such, next-generation TB vaccines should aim to boost protective immunity in BCG-primed humans. Given this, the boosting efficacy of Tri:ChAd:TB vaccine was examined. To this end, mice were either subcutaneously (s.c.) immunized with BCG alone (BCG), or were subsequently i.n. boosted 4 weeks post-BCG with Tri:ChAd:TB (BCG Tri). A set of BCG-primed mice were i.n. boosted with Mono:ChAd:TB (BCG Mono) as a comparison. Animals were sacrificed 2 weeks post-boost and mononuclear cells were isolated from the airway lumen (BAL) and lung tissue (FIG. 2A). Antigen-specific CD4⁺ and CD8⁺ T cells were quantified by flow cytometry for expression of IFNγ following ex vivo stimulation with either crude BCG antigens, or recombinant Ag85A, TB10.4, or rpfB proteins. Parenteral BCG priming alone did not induce airway luminal CD4⁺ and CD8⁺ T cell responses (FIG. 2B), whereas it induced a level of crude BCG antigen reactive CD4⁺ and CD8⁺ T cell responses (BCG-specific) in the lung (FIG. 8A). i.n. Mono:ChAd:TB boost-immunization of BCG-primed hosts (BCG Mono) markedly increased BCG-specific CD4⁺ T cells in the airway lumen (BAL) and lung (FIG. 2B & FIG. 8A). It also increased BCG-specific CD8⁺ T cells in the BAL (FIG. 2B). In comparison, Tri:ChAd:TB respiratory mucosal boosting markedly increased both CD4⁺ and CD8⁺ T cell responses in the airway lumen and lung and the magnitude of such responses were significantly greater than that induced by the monovalent counterpart (FIG. 2B & FIG. 8A). Functionality of airway luminal and lung BCG-specific CD4+ and CD8+T cells induced by either vaccine in BCG-primed hosts was comparable (FIG. 2C & FIG. 8B). Of interest, the majority of airway luminal and lung BCG-specific CD4⁺ T cells were bifunctional (IFNγ⁺ TNFα⁺), whereas BCG-specific CD8⁺ T cells were either single cytokine producers (IFNγ⁺) or bifunctional (IFNγ⁺ TNFα⁺) (FIG. 2C and FIG. 8B). Moreover, i.n. boosting with either vaccine markedly increased bifunctional (IFNγ⁺ TNFα⁺) BCG-specific CD4 T cells in the lung of BCG-primed hosts (FIG. 8B).

Given that Ag85A, TB10.4, and rpfB-specific T cell responses were detected in BCG vaccinated hosts (Hervas-Stubbs et al., 2006; Metcalfe et al., 2016; Mukamolova et al., 2002), it was assessed whether or not these responses were boosted in the airway lumen (BAL), lung and spleen of BCG-primed hosts when they were i.n. immunized with Tri:ChAd:TB (BCG/Tri). A set of BCG-primed animals were i.n. immunized with Mono:ChAd:TB (BCG/Mono) as a comparison (FIG. 2A). Of note, s.c. BCG priming alone did not induce airway luminal CD4⁺ and CD8⁺ T cell responses (FIG. 2B). Boosting BCG-primed hosts i.n. with either Tri:ChAd:TB vaccine or the monovalent counterpart markedly increased CD4⁺ and CD8⁺ T cells reactive to all three antigens in the airway lumen (FIG. 2D/E). Tri:ChAd:TB immunization differed significantly from its monovalent counterpart in its capacity to markedly boost TB10.4 and rpfB-specific CD4⁺ and CD8⁺ T cells in BCG-primed hosts, with a 2-fold increase in TB10.4 and rpfB reactive CD4⁺ T cells and 4- and 20-fold increase in TB10.4 and rpfB reactive CD8⁺ T cells, respectively, compared to those boosted with monovalent counterpart (FIG. 2D/E). Monovalent counterpart induced significantly increased Ag85A reactive CD8⁺ T cells than Tri:ChAd:TB vaccine in the airway lumen (FIG. 2E). Similar trends were observed in the lung (FIG. 8C/D). Of interest, intranasal Tri:ChAd:TB immunization in parenteral-BCG primed host not only boosted the respiratory mucosal BCG-specific responses, but also boosted such responses remotely in the spleen (FIG. 2F). Such boosting effect was not evident following intranasal boosting with monovalent counterpart.

The above data together indicate that Tri:ChAd:TB boosting potently enhances BCG—as well as multi-stage antigen-specific CD4 and CD8 T cell responses on the respiratory mucosal surfaces and at peripheral sites.

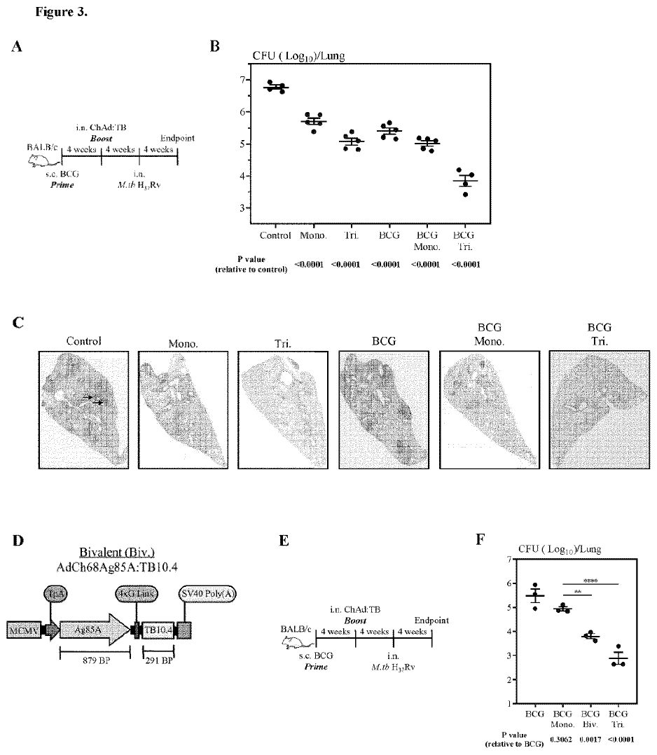

Intranasal immunization with multi-stage ChAd:TB vaccine provides enhanced protection against pulmonary M.tb infection over its monovalent counterpart—To investigate whether the induction of multi-stage antigens-specific responses on the respiratory mucosal surfaces in naïve and parenteral BCG-primed hosts could lead to improved protection against pulmonary TB, naive mice or 4 week s.c. BCG-primed mice were i.n. immunized with either Tri:ChAd:TB or its monovalent counterpart. As controls, a group of mice was left unvaccinated (control) or subcutaneously immunized with BCG for 8 weeks. All mice were infected with virulent M.tb (H₃₇Rv), and sacrificed 4 weeks post-infection (FIG. 3A). Relative levels of protective efficacy in the lung were assessed by enumerating mycobacterial burden using a solid agar colony forming unit (CFU) assay. Formalin fixed lung sections were subjected to Hematoxylin and Eosin (H&E) staining and histopathological analysis. BCG priming (BCG) and intranasal Mono:ChAd:TB (Mono) as a standalone or BCG-Mono:ChAd:TB (BCG Mono) immunization significantly enhanced protection, causing 1.0, 1.3 and 1.8 log₁₀M.tb colony-forming unit (CFU) reduction, respectively, compared to control (FIG. 3B) (Horvath et al., 2012; Jeyanathan et al., 2015). Of interest, correlating with its ability to induce multi-stage antigen-specific T cell immunity in naïve hosts (Tri) (FIG. 1) and markedly boosting BCG-specific CD4⁺ and CD8⁺ T cells that were also specific to chronic/latent antigens TB10.4 and rpfB (BCG Tri) (FIG. 2A-D), intranasal Tri:ChAd:TB immunization further improved protection causing 1.7 log and 2.9 log reduction in M.tb CFU, respectively (FIG. 3B). Protection rendered by immunization with Tri:ChAd:TB alone or boosting BCG-induced immunity were significantly greater than the protection rendered by its monovalent counterpart, causing further reduction in bacterial burden by 4 and 13-fold, respectively (Mono. vs Tri. p=0.003 and BCG Mono vs BCG Tri p<0.0001).

In keeping with markedly improved protection rendered by Tri:ChAd:TB either as a stand-alone (Tri.) or BCG booster (BCG Tri.) immunization, remarkably reduced lung immunopathology was noted in these animals (FIG. 3C). Although mono:ChAd:TB either as a stand-alone (Mono.) or BCG booster (BCG Mono.) reduced overall immunopathology compared to unvaccinated (Control) and BCG immunized (BCG) hosts, it did not prevent occurrence of small focal areas of granulomatous inflammation (black arrows). In stark contrast, naïve and BCG-primed hosts that received single dose of i.n. Tri:ChAd:TB immunization displayed only a mild inflammatory infiltrates located in the parabronchial and perivascular regions. Collectively, the above data indicate that encoding the chronic and latent TB antigens in the vaccine construct can further improve the potency of the vaccines against pulmonary tuberculosis.

Inclusion of chronic/latent antigens in vaccine design provides added protection against pulmonary M.tb infection over the vaccine expressing only an acute antigen—Having demonstrated the superior protective efficacy of Tri:ChAd:TB vaccine over its monovalent counterpart, the relative contribution of chronic (TB10.4) and latent (rpfB) TB antigens encoded in Tri:ChAd:TB was assessed for its robust protection. To do so, another vaccine was produced using the same replication-deficient chimpanzee type 68 adenovirus vector that was used for the construction of Tri:ChAd:TB vaccine. A bivalent vaccine to encode Ag85A and TB10.4 was constructed, hereafter referred to as Biv:ChAd:TB vaccine (FIG. 3D). To investigate the relative role of TB10.4 and rpfB-specific immunity in protection against pulmonary TB, 4 week s.c. BCG-primed mice were i.n. immunized with an equal dose of either Mono:ChAd: TB, Biv:ChAd: TB, or Tri:ChAd: TB. A group of mice were s.c. immunized with BCG for 8 weeks. All groups of mice were infected with virulent M.tb (H₃₇Rv), and sacrificed 4 weeks post-challenge (FIG. 3E). While boosting with monovalent vaccine marginally reduced the lung mycobacterial burden compared to BCG, both bivalent and trivalent vaccines significantly reduced the lung mycobacterial burden (FIG. 3F). Of interest, the protection rendered by Biv:ChAd:TB vaccine was significantly greater than monovalent counterpart but significantly inferior to the protection rendered by Tri:ChAd:TB. These data indicate that the remarkable enhanced protection provided by Tri:ChAd:TB over its monovalent counterpart is intrinsically linked to the inclusion of chronic/latent antigens, TB10.4, and rpfB in the vaccine construct and rpfB being important for the best level of protection.

Inclusion of a latent antigen in vaccine design significantly reduces non-culturable, persistent M.tb bacilli following antibiotic cessation—It is now well known that under immunological and pharmacological pressure, M.tb bacilli can acquire a non-replicating state, hereafter referred to as persisters. Having determined that boosting BCG-primed hosts with intranasal Tri:ChAd:TB vaccine induced robust airway luminal and lung CD4 and CD8 T cell immunity against rpfB antigen (FIG. 2B/D & FIG. 8C/D) which was associated with best level of protection against pulmonary TB (FIG. 3B/F), it was next investigated whether such outcome was because rpfB-specific immunity hindered development of persisters and as such prevented immune invasive strategy of M.tb bacilli. To address this, an antibiotic therapy model of pulmonary tuberculosis was adapted to trigger development of persisters in vaccinated animals. To this end, 4-wk BCG-primed animals were intranasally boosted with either Mono or Biv or Tri:ChAd:TB vaccine. All groups of mice were infected with virulent M.tb (H37Rv) at 4-wk post booster immunization. As the control, some mice were subcutaneously immunized with BCG and at 8-wks post-immunization were infected with M.tb. At 4-wks after infection all animals were treated with triple antibiotic therapy for 2-wks. Animals were sacrificed at 4-wks after cessation of antibiotic therapy and lungs were collected for M.tb enumeration by conventional solid agar CFU assay (FIG. 4A). As, expected intranasal boosting with mono, biv and trivalent vaccines significantly reduced bacterial burden in the lung compared to control animals (BCG). Importantly, Tri:ChAd:TB booster immunization rendered the best level reduction (~3 log) in the bacterial burden (FIG. 4B).

In the same experimental setup, the most probable number (MPN) assay was performed that is based on bacterial growth in liquid media to enumerate the actively replicating and non-replicating dormant (persisters) M.tb bacilli. Lung homogenates were cultured in liquid media supplemented with M.tb culture filtrate to resuscitate persisters into an active replication state and allows enumeration of both actively replicating bacilli and persisters. Another aliquot of the same sample was cultured in liquid media without supplementation as a control which allows enumeration of only actively replicating bacilli (FIG. 4C). The presence of non-replicating persisters were determined by comparing absolute MPN number between M.tb culture filtrate treated and untreated samples. Of interest, the number of actively growing and non-replicating persisters were significantly reduced compared to control (BCG) only in animals that were boosted with Tri:ChAd:TB vaccine (BCG Tri) (FIG. 4D). Importantly, the number of persisters detected in these animals were almost at undetectable levels. Resuscitation index (RI) which is a numerical representation of relative abundance of persisters-to-actively replicating mycobacteria indicated that indeed Tri:ChAd:TB booster immunization effectively hindered the development of persisters (FIG. 4D). In stark contrast, monovalent counterpart fortified the development of persisters as indicated by the highest RI among the boosting vaccines.

These data together support the role of rpfB-specific immunity on the respiratory mucosal surface induced by the present multi-stage vaccine design and respiratory mucosal immunization for effective control of pulmonary TB by hindering development of non-replicating persisters.

Intranasal therapeutic immunization with multi-stage ChAd:TB vaccine significantly reduces nonculturable, persistent M.tb bacilli—Immunotherapy adjunct to antibiotic therapy has the potential to significantly accelerate disease control and shorten the duration of conventional treatment. However, the relative impact of adjunct immunotherapy, particularly the therapeutic respiratory mucosal immunization on driving development of non-replicating persisters was unknown. Given the superior ability of intranasal Tri:ChAd:TB immunization to prophylactically hinder development of non-replicating persisters (FIG. 4D), it was determined whether or not therapeutic immunization with Tri:ChAd:TB accelerates mycobacterial clearance and reduces establishment of persisters. An immunotherapy model as described previously (Afkhami et al., 2019. J Infect Dis 220, 1355-1366) was adapted. Briefly, 4-wks after M tb infection, mice were treated with triple antibiotic cocktail for 4-wks. Groups of mice were immunized intranasally either with Tri:ChAd:TB vaccine (Tri) or monovalent counterpart (Mono) at 4-wks post-cessation of antibiotic therapy. Some mice were left unvaccinated. All groups of mice were sacrificed at 4-wks post immunotherapy (FIG. 4E). Bacterial burden in the lung was enumerated using MPN assay as described above (FIG. 4C). Immunotherapy with monovalent vaccine did not reduce actively replicating M.tb bacilli as measured by MPN assay, whereas Tri:ChAd:TB vaccine significantly reduced actively replicating M tb bacilli in the lung compared to unvaccinated control (FIG. 4F). Non-replicating persisters count was very small in all groups and did not differ between unvaccinated and Tri:ChAd:TB vaccine treated hosts. Interestingly, immunotherapy with the monovalent vaccine promoted development of persisters as indicated by significantly increased persisters count and higher RI value compared to unvaccinated counterparts. Together, these data suggest that selection of immunotherapy using viral-vectored vaccines adjunct to antibiotic therapy should be done with caution.

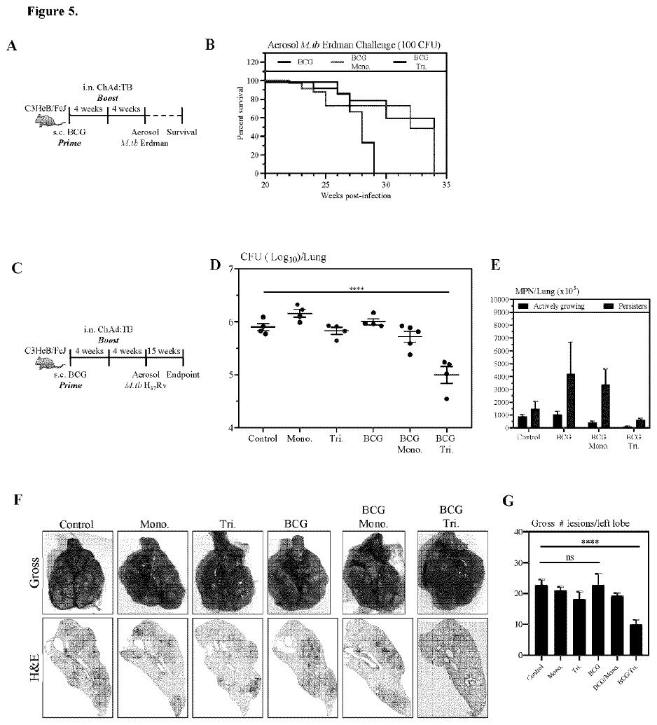

Intranasal immunization with multi-stage ChAd:TB vaccine provides enhanced protection against pulmonary infection with a highly virulent M.tb strain in a susceptible murine model—The data to this point indicates that multi-stage ChAd:TB vaccine is superior to its monovalent counterpart which expresses only an acute stage antigen and such robust protection was linked to latent/dormant (rpfB) antigen-specific immunity induced by the vaccine which played a role in controlling of non-replicating persisters. These outcomes were seen in a relatively resistant BALB/c model of tuberculosis infected with a laboratory virulent strain of M.tb H₃₇Rv. This model is limited in mimicking characteristic of pulmonary tuberculosis in humans. Thus, the protective efficacy of the multi-stage ChAd:TB vaccine was next assessed in a more susceptible mouse model of TB, C3HeB/FeJ mice (hereafter referred to as FeJ mice), which mimic the human disease. Thus, parenteral BCG-primed FeJ mice were intranasally boosted with Tri:ChAd:TB or the monovalent counterpart and at 4-wks post-boost challenged with low-dose (100 CFU) aerosol M.tb (Erdman) (FIG. 5A). As control, some mice were BCG-primed for 8-wks and infected. An improved survival was observed in boosted animals (BCG/Mono and BCG/Tri) over the control animals (BCG) (FIG. 5B). Although survival rate did not differ significantly between mice boosted with monovalent and Tri:ChAd:TB vaccines, Tri:ChAd:TB-boosted animals tend to succumb to infection a little later than those boosted with monovalent vaccine.

The capacity of the multi-stage ChAd:TB vaccine to control bacterial burden in the stringent murine model of TB described above was assessed by infection with low-dose (100 CFU) aerosol M.tb H₃₇Rv. Mice were sacrificed 15-wks post-infection (FIG. 5C) and bacterial burden was quantified by conventional sold agar CFU assay in all groups of mice (FIG. 5D). Lung homogenates from selected groups were subjected to MPN assay (FIG. 5E). Compared to control, lung bacterial burden was significantly reduced only in the animals that were boosted with Tri:ChAd:TB vaccine (~10-fold reduction) (FIG. 5D). Though no significant differences in the counts of replicating and non-replicating dormant M.tb bacilli was observed between groups by MPN assay, compared to control, Tri:ChAd:TB vaccine-boosted animals harboured the least number of both replicating and non-replicating M.tb bacilli among all groups (FIG. 5E). Importantly, gross pathological indices reflected by arbitrary scores based on number of lung nodules indicated significant reduction in lung injury in animals that received Tri:ChAd:TB as a BCG-boost immunization (gross lung images and FIG. 5G). Similarly, significant reduction in granulomatous lesions were observed in the lung of BCG-primed Tri:ChAd:TB vaccine boosted animals (FIG. 5G).

Taken together, these data indicate that even in a most stringent model of pulmonary tuberculosis, Tri:ChAd:TB vaccine, but not the monovalent counterpart as a BCG booster, provides robust protection by controlling bacterial burden and associated lung immunopathology.

Intranasal immunization with multi-stage ChAd:TB vaccine provides enhanced protection against pulmonary M.tb infection in a humanized mouse model—Given the protective efficacy observed with the multi-staged ChAd:TB vaccine, the efficacy of this vaccine in humanized-mouse model was determined to assess its potential clinical relevance. Humanized mice were generated as previously described (Yao et al., 2017. J Infect Dis 216, 135-145). Briefly, irradiated newborn NOD-Rag1^{tm1Mom}Il2rg^{tm1Wjl} (NRG) mice were reconstituted with CD34⁺ hematopoietic stem cells enriched from human cord blood. Engraftment was confirmed by flow cytometry at 12 weeks post-reconstitution (Table 1).

TABLE 1

| Group | % HuCD45 | % HuCD3 | % HuCD4 | % HuCD8 |
|---|---|---|---|---|
| Control | 25.50 | 55.70 | 54.90 | 32.60 |
| Tri:ChAd:TB | 15.80 | 52.80 | 46.50 | 37.40 |

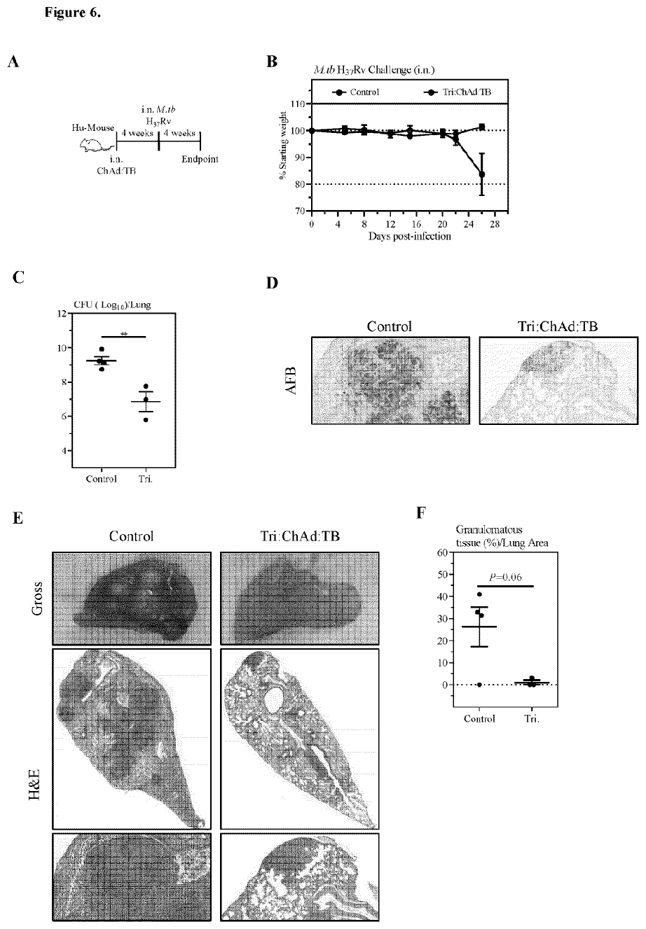

Animals were randomized to either remain unvaccinated, or be i.n. immunized with Tri:ChAd:TB vaccine. Animals were challenged with M.tb H₃₇Rv at 4-wks post-immunization, and monitored for weight loss as an indices of TB disease. (FIG. 6A). In the same experiment pre-defined sacrifice time was set at 4-wks post-infection to quantify lung mycobacterial burden and evaluate lung injury (FIG. 6A). Given the highly susceptible nature of Hu-mice to TB, considerable weight loss among unvaccinated animals, on average ~20%, was observed. In contrast, at this timepoint all intranasal Tri:ChAd:TB immunized animals did not lose weight (FIG. 6B). In congruence with these clinical observations, a significant reduction in mycobacterial burden was observed in the lung as determined by CFU assay in Tri:ChAd:TB immunized mice (2.40 log reduction compared to control) (FIG. 6C). Enhanced bacterial control in immunized mice were further supported by markedly reduced densities of acid-fast bacilli (AFB) in the microscopic lung sections (FIG. 6D). Additionally, lungs from immunized animals showed remarkably reduced gross pathological changes (FIG. 6D, Gross) and microscopic granulomatous lesions (FIG. 6D/E). Collectively, the above data indicate that i.n. immunization with a multi-stage ChAd:TB vaccine can provide robust protection against TB in a clinically translatable and highly susceptible humanized mouse model.

### Discussion

A vaccine is herein described that elicits immunity specific for multiple antigens expressed by M.tb bacilli during its lifecycle. The Tri:ChAd:TB vaccine administered via the respiratory mucosal route elicits immunity against acute infection-associated antigen, Ag85A, acute/chronic infection antigen, TB10.4, and dormant/latent antigen, rpfB, and provides remarkably enhanced protection against two virulent strains of M.tb in three separate murine models including a clinically relevant, highly susceptible humanized mouse model. It is further shown that this vaccine strategy could accelerate TB control adjunct to TB antibiotic therapy. Thus, the present vaccine exhibits effective prophylactic and therapeutic capacity targeting both replicating and non-replicating dormant M tb bacilli.

Importantly, Tri:ChAd:TB vaccine provided the best levels of protection compared to its monovalent and bivalent counterparts as a standalone or BCG booster vaccine (FIG. 3). Such enhanced protection was associated with its ability to better control not only the replicating but also the non-replicating dormant M.tb bacilli (FIG. 4D). Of note, the monovalent counterpart, despite its ability to induce comparable Ag85A reactive T cell immunity to that induced by Tri:ChAd:TB, was unable to effectively control reactivation of dormant bacilli induced following antibiotic therapy, indicating that the vaccine strategies solely targeting the early secreted antigens of M.tb are not an effective prophylactic/immunotherapeutic approach. This line of observation is further supported by the immunogenicity induced by these vaccines in BCG-primed hosts (FIG. 2). While Tri:ChAd:TB vaccine boosted comparable levels of Ag85A reactive T cells to that boosted by monovalent vaccine, frequencies of TB10.4 and rpfB reactive T cells were much greater in Tri:ChAd:TB vaccine boosted hosts, indicating the critical role played by chronic/latent antigen reactive T cells in effective protection. Of note, TB10.4 and rpfB reactive T cells were also detected following monovalent boost despite the fact that these antigens are not expressed by BCG, suggesting cross-reactive nature of BCG-induced immunity.

The present multivalent vaccine is a first-of-its-kind multi-stage vaccine which is suitable for respiratory mucosal administration. The poor efficacy of vaccines such as BCG or MVA85A against pulmonary TB may stem from the fact that vaccine-derived immunity is confined to the periphery following parenteral route of vaccination. Thus, the present viral-vectored multi-stage TB vaccine which is amenable for respiratory mucosal administration is effective to induce multifunctional tissue-resident memory T cells on the respiratory mucosa. Indeed, it has been observed that mucosal antigen-reactive T cells against all three antigens expressed by the present vaccine displayed characteristics of resident memory and were maintained for a long time following immunization such that rpfB reactive mucosal-resident T cells effectively controlled/eradicated persisters (FIG. 1I & FIG. 4C).

The current study has for the first time examined the heterogenous populations (replicating and non-replicating dormant) of M.tb bacilli in the lung as a marker of protective efficacy of a multi-stage vaccine in a variety of murine models including highly susceptible FeJ mouse and humanized mouse models. Although the presence of M.tb persisters has been demonstrated in sputum samples of TB patients and following antibiotic therapy, their analysis in the lung of pre-clinically vaccinated animals has never been carried out before. The present data indicate that a heterogeneous population of M tb bacilli is present in the lung of vaccinated hosts and reveal that rpfB reactive T cells on the respiratory mucosal surface can eradicate/control persisters (FIG. 4D & FIG. 5E). Apart from proper assessment of heterogenous M.tb populations in vaccine evaluation studies, the validity of the experimental animal models of pulmonary tuberculosis is a consideration. Genetic heterogeneity of mice and the dose and virulence of M.tb used for infection can all influence the protective outcomes and clinical relevance in vaccine evaluation studies. Using the most stringent FeJ murine model of tuberculosis, which develops necrotizing granuloma more consistent with human tuberculosis disease, it is herein demonstrated that the present multi-stage viral-vectored vaccine can prolong the survival of M.tb infected hosts, which was associated with markedly reduced replicating and non-replicating dormant M.tb bacilli and necrotizing granuloma in the lung (FIG. 5). The present vaccine conferred protective efficacy in FeJ mice following both Erdman and H37Rv aerosolized M.tb challenge. In this study, BCG as a standalone vaccine failed to protect FeJ mice, different from its protective effects in BALB/c mice. This is likely related to the longer time post-infection (15-wks) chosen to study the protective efficacy as compared to other studies which mostly studied protective efficacy at 4-wks post-vaccination (Henao-Tamayo et al., 2015) and that C3HeB/FeJ mice are H2-k restricted, thus incapable of presenting several CD4 T cell antigens. In this regard, intranasal immunization in BCG-primed hosts strongly boosted CD4 T cell responses to TB10.4 and rpfB (FIG. 2E), which could have contributed to the markedly enhanced protection. Furthermore, the observation that intranasal immunization also significantly protected the highly susceptible humanized animals from pulmonary TB disease further highlights the efficacy of this multi-stage TB vaccine strategy.

In summary, it is shown for the first time that the multi-stage viral-vectored next-generation tuberculosis vaccine delivered via respiratory mucosal route remarkably enhances protection against pulmonary tuberculosis in three separate murine models including a highly clinically relevant humanized mouse model. Of importance, for the first time it is shown that this vaccine is able to expand protective efficacy by controlling both replicating and non-replicating dormant M.tb bacilli in the lung, and accelerates TB control adjunct to TB antibiotic therapy.

The relevant content of references referred to herein is incorporated herein by reference.

1. A transgene that encodes a multivalent tuberculosis vaccine encoding a first acute mycobacterial infection-associated antigen, a second acute/chronic mycobacterial infection-associated antigen, and a third dormant/latent resuscitation mycobacterial infection-associated antigen.

2. A trivalent vaccine comprising a transgene as defined in claim 1.

3. The transgene of claim 1, or the vaccine of claim 2, wherein the acute infection stage antigen is selected from Ag85A, Ag85B and Ag85C antigens, the acute/chronic infection-associated antigen is selected from TB10.4, ESAT6, CFP-10 and Esp proteins, and the dormant/latent resuscitation infection-associated antigen is selected from RpfA, RpfB, RpfC, RpfD and RpfE.

4. The transgene of claim 1, or the vaccine of claim 2, wherein the acute infection-associated antigen is Ag85A, the acute/chronic infection-associated antigen is TB10.4, and the dormant/latent resuscitation infection-associated antigen is RpfB.

5. The trivalent vaccine of any one of claims 2-4, which is prepared for administration via the respiratory tract.

6. The trivalent vaccine of any one of claims 2-5, which is a viral vaccine.

7. The trivalent vaccine of any one of claims 2-6, which is an adenoviral vaccine.

8. The trivalent vaccine of any one of claims 2-5, which is a DNA or mRNA vaccine.

9. The trivalent vaccine of claim 8, which is complexed with an adjuvant.

10. The trivalent vaccine of any one of claims 2-5, which is a protein vaccine.

11. The transgene of any one of claims 1 or 3-4, or the vaccine of any one of claims 2-10, wherein the transgene comprises an N-terminal signal sequence linked to the first antigen which is linked to the second antigen which is linked to the third antigen.

12. A method of vaccinating a mammal against a mycobacterial infection comprising administering a trivalent vaccine as defined in any one of claims 2-11 to a mammal.

13. The method of claim 12, wherein the vaccine is administered via the respiratory tract.

14. The method of claim 12 or 13, wherein the vaccine is administered prophylactically.

15. The method of claim 12 or 13, wherein the mammal has a mycobacterial infection.

16. The method of any one of claims 11-15, wherein the mammal has been vaccinated with Bacillus Calmette-Guerin.

17. The method of claim 15, additionally comprising the step of administering to the mammal an antibiotic useful to treat a mycobacterial infection.

18. The method of any one of claims 12-17, wherein administration of the vaccine results in a reduction of both replicating and non-replicating mycobacteria.

19. The method of claim 13, wherein the vaccine results in an immune response in the lung and remote from the lung.

20. A kit comprising a vaccine as defined in any one of claims 2-11 and a nebulizer.
